(19) United States
(12) Patent Application Publication
(10) Pub. No.: US 2025/0213365 A1
(43) Pub. Date: Jul. 3, 2025

(54) INFINITELY ADJUSTABLE AUGMENT ANGLE BASEPLATE

(71) Applicant: Zimmer, Inc., Warsaw, IN (US)

(72) Inventors: Ravikumar Varadarajan, Warsaw, IN (US); Steven E. Stump, Goshen, IN (US)

(21) Appl. No.: 18/936,524

(22) Filed: Nov. 4, 2024

Related U.S. Application Data
(60) Provisional application No. 63/616,306, filed on Dec. 29, 2023.

(51) Int. Cl. A61F 2/40; A61F 2/30

(52) U.S. Cl. CPC A61F 2/4081; A61F 2/4059; A61F 2002/3054; A61F 2002/4018

(57) ABSTRACT

A system for an anatomical total shoulder arthroplasty or reverse total shoulder arthroplasty can include an infinitely adjustable augment angle baseplate, an implant, a baseplate, an engagement member, and a prosthetic head. The infinitely adjustable augment angle baseplate can be configured to be secured to a first bone of a patient. The implant can be insertable into a second bone of a patient. The baseplate can be insertable into the implant and configured to be attached to the second bone of the patient. The engagement member can be couplable to either of the infinitely adjustable augment angle baseplate or the baseplate. The prosthetic head can be couplable to the other of the infinitely adjustable augment angle baseplate or the baseplate. The prosthetic head can be engageable with the engagement member to form a prosthetic joint between the first bone and the second bone.

### CLAIM OF PRIORITY

This patent application claims the benefit of priority, under 35 U.S.C. Section 119 (e), to Ravikumar Varadarajan U.S. Patent Application Ser. No. 63/616,306, entitled “INFINITELY ADJUSTABLE AUGEMENT ANGLE BASEPLATE,” filed on Dec. 29, 2023, which is hereby incorporated by reference herein in its entirety.

### TECHNICAL FIELD

Examples described herein generally relate to a baseplate for a prosthetic implant. More specifically examples described herein generally relate to an infinitely adjustable augment angle baseplate for a prosthetic implant.

### BACKGROUND

The shoulder joint is a complex joint with the scapula, clavicle, and humerus all coming together to enable a wide range of movement, at least in a properly functioning joint. In a properly functioning shoulder joint, the head of the humerus fits into a shallow socket in the scapula, typically referred to as the glenoid. Articulation of the shoulder joint involves movement of the humeral head in the glenoid, with the structure of the mating surfaces and surrounding tissues providing a wide range of motion.

The shoulder joint can undergo degenerative changes caused by various issues, such as rheumatoid arthritis, osteoarthritis, rotator cuff arthroplasty, vascular necrosis, or bone fracture. When severe joint damage occurs, and no other means of treatment is found to be effective, a total, partial, or reverse shoulder replacement or reconstruction may be necessary. Total shoulder replacements can involve a humeral prosthetic, including a stem and a head portion used to replace the natural humeral head. Total shoulder replacements will also typically include resurfacing of the glenoid with a prosthetic implant. The glenoid implant will generally include an articulating cup shaped to receive the prosthetic humeral head. A reverse shoulder replacement (arthroplasty) involves a different set of humeral and glenoid replacement prosthetics. In a reverse shoulder replacement, the humeral component includes a cup-shaped articular surface attached to a stem implanted into the humerus, while a spherical glenoid component is used to provide an articular surface for the humeral cup.

### SUMMARY

In examples, a baseplate can include an infinitely adjustable augment angle for a prosthetic implant. The baseplate can include a first portion having a first support surface, a first anchoring surface opposite the first support surface, and a first fastener aperture extending through the first portion from the first support surface to the first anchoring surface. The baseplate can also include a second portion pivotably attached to the first portion, the second portion including a second support surface, a second anchoring surface opposite the second support surface, and a second fastener aperture extending through the second portion from the second support surface to the second anchoring surface. Pivoting the second portion relative to the first portion can alter the infinitely adjustable augment angle.

In examples, a system for an anatomical total shoulder arthroplasty or reverse total shoulder arthroplasty can include an infinitely adjustable augment angle baseplate, an implant, a baseplate, an engagement member, and a prosthetic head. The infinitely adjustable augment angle baseplate can be configured to be secured to a first bone of a patient. The implant can be insertable into a second bone of a patient. The baseplate can be insertable into the implant and configured to be attached to the second bone of the patient. The engagement member can be couplable to either of the infinitely adjustable augment angle baseplate or the baseplate. The prosthetic head can be couplable to the other of the infinitely adjustable augment angle baseplate or the baseplate. The prosthetic head can be engageable with the engagement member to form a prosthetic joint between the first bone and the second bone.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 is a perspective view of an example of a prosthetic implant including an infinitely adjustable augment angle baseplate.

FIG. 2 is an exploded view of an example of a prosthetic implant including an infinitely adjustable augment angle baseplate.

FIG. 3 is an exploded view of an example of an infinitely adjustable augment angle baseplate.

FIG. 4 is a perspective view of an example of an infinitely adjustable augment angle baseplate.

FIG. 5 is a side view of an example of an infinitely adjustable augment angle baseplate.

FIG. 6 is a side view of an example of an infinitely adjustable augment angle baseplate including an adjusted infinitely adjustable augment angle.

FIG. 7 is a partial cross-sectional view of an example of an infinitely adjustable augment angle baseplate including an adjusted infinitely adjustable augment angle.

### DETAILED DESCRIPTION

Patients with eroded glenoids, which are the socket portions of the shoulder joint, often experience significant pain and limited functionality. The erosion of the glenoid can be caused by a range of factors, such as osteoarthritis, trauma, or repetitive strain. Traditional treatment options, such as bone grafts or partial shoulder replacements, may not always provide satisfactory outcomes, leaving patients with continued discomfort and restricted movement. In such cases, augmented glenoid implants offer a compelling solution. These implants are designed to reconstruct and reinforce the damaged glenoid, providing stability and restoring normal joint mechanics. By improving joint function and relieving pain, augmented glenoid implants aim to enhance the quality of life for patients with eroded glenoids.

The currently available augmented glenoid implants are available in discrete augment angles and may result in excessive bone removal and may not always provide an ideal fit for the patient, leading to implant loosening, instability, and decreased function. To overcome these issues, an infinitely variable augment angle baseplate implant is envisioned. This technology allows for less bone removal, minimizes inventory, and reduces surgical error.

The infinitely adjustable augment angle baseplate described herein can provide a new option for patients with eroded glenoids. Unlike currently available implants with discrete augment angles, the described design can offer a flexible solution tailored to individual patient needs. By allowing infinite angle adjustments within a range of augment angles, surgeons can precisely customize the implant's position and orientation to achieve optimal joint alignment and stability. This enhanced level of customization can ensure a more natural biomechanical fit and reduce the risk of implant-related complications. Moreover, the ability to fine-tune the augment angles can enable surgeons to address complex cases and accommodate variations in patients' anatomy, improving the overall success rates and long-term outcomes of augmented implant procedures.

In addition to the benefits of infinite augment angles, the introduction of a single stock-keeping unit (hereinafter “SKU”) customizable implant for augmented procedures can streamline inventory management and decrease surgical errors. Unlike traditional implants that are offered at discrete augment angles and require a wide range of inventory, the pivot hinge design implant can eliminate the need for multiple implant options. With a single SKU implant, hospitals and healthcare providers can reduce their inventory carrying costs and simplify the logistics associated with ordering and storing different implant variations.

Furthermore, the customizable nature of the implant can help decrease surgical errors by providing surgeons with the flexibility to finetune the augment angles intraoperatively, ensuring precise placement and alignment. This adaptability can enhance surgical efficiency, improve patient outcomes, and contribute to a more cost-effective healthcare system.

In examples, a baseplate can include an infinitely adjustable augment angle for a prosthetic implant. The baseplate can include a first portion having a first support surface, a first anchoring surface opposite the first support surface, and a first fastener aperture extending through the first portion from the first support surface to the first anchoring surface. The baseplate can also include a second portion pivotably attached to the first portion, the second portion including a second support surface, a second anchoring surface opposite the second support surface, and a second fastener aperture extending through the second portion from the second support surface to the second anchoring surface. Pivoting the second portion relative to the first portion can alter the infinitely adjustable augment angle.

In examples, a system for an anatomical total shoulder arthroplasty or reverse total shoulder arthroplasty can include an infinitely adjustable augment angle baseplate, an implant, a baseplate, an engagement member, and a prosthetic head. The infinitely adjustable augment angle baseplate can be configured to be secured to a first bone of a patient. The implant can be insertable into a second bone of a patient. The baseplate can be insertable into the implant and configured to be attached to the second bone of the patient. The engagement member can be couplable to either of the infinitely adjustable augment angle baseplate or the baseplate. The prosthetic head can be couplable to the other of the infinitely adjustable augment angle baseplate or the baseplate. The prosthetic head can be engageable with the engagement member to form a prosthetic joint between the first bone and the second bone.

The above discussion is intended to provide an overview of subject matter of the present patent application. It is not intended to provide an exclusive or exhaustive explanation of the invention. The description below is included to provide further information about the present patent application.

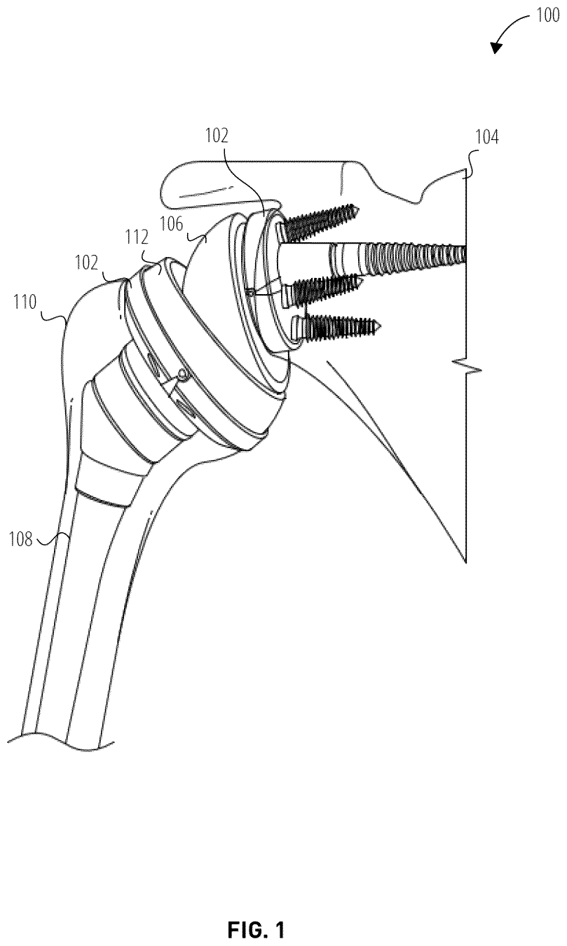

FIG. 1 illustrates a perspective view of an example of a prosthetic implant 100 including an infinitely adjustable augment angle baseplate 102. The prosthetic implant 100 can be an anatomical total shoulder arthroplasty, reverse total shoulder arthroplasty (as shown in FIG. 1), or any other stemmed or stemless implant used to replace a joint in the human body. The prosthetic implant 100 can include an infinitely adjustable augment angle baseplate 102, which can be configured to be installed in the first bone 104 of the patient, an implant 108, which can be implantable into the second bone 110 of the patient, and an engagement member 112.

The infinitely adjustable augment angle baseplate 102 can be infinitely adjustable within a pre-defined range as set by the physical limitations of the infinitely adjustable augment angle baseplate 102. As such, there are no set adjustments and the augment angle of the infinitely adjustable augment angle baseplate 102 can be any angle between a maximum augment angle and a minimum augment angle as defined by the infinitely adjustable augment angle baseplate 102. As shown in FIG. 1, a first of the infinitely adjustable augment angle baseplate 102 can be installed in the implant 108 and attached to the second bone 110 and a second of the infinitely adjustable augment angle baseplate 102 can be attached to the first bone 104. In examples, the prosthetic implant 100 can include a standard baseplate (e.g., not the infinitely adjustable augment angle baseplate 102) coupled to the implant 108 and attached to the second bone 110.

As shown in FIG. 1, the infinitely adjustable augment angle baseplate 102 can be installed into either the first bone 104 or the second bone 110. When installed in the first bone 104, the infinitely adjustable augment angle baseplate 102 can be directly attached to the first bone 104 and when attached to the second bone 110, the infinitely adjustable augment angle baseplate 102 can be installed into the implant 108 and attached to the second bone 110. As such, the infinitely adjustable augment angle baseplate 102 can be configured to receive, inserted into, or otherwise coupled to the artificial head component 106, the engagement member 112, or other prosthetic heads or articulating members of prosthetic joints.

As shown in FIG. 1, the implant 108 can be a stemmed implant. The implant 108 can also be a stemless implant. Either the stemmed or stemless version of the implant 108 can be configured to be implanted into the second bone 110. The implant 108 can be elongated to increase the surface area between the implant 108 and the second bone 110, which can improve the support of the implant 108 after implantation into the second bone 110. The implant 108 can be configured to receive the infinitely adjustable augment angle baseplate 102.

The engagement member 112 can be configured to engage with the artificial head component 106 to form a joint after insertion of the prosthetic implant 100 into the patient. In an anatomical total shoulder arthroplasty, the engagement member 112 can be connected to the infinitely adjustable augment angle baseplate 102 attached to the first bone 104. In a reverse total shoulder arthroplasty, the engagement member 112 can be coupled to the infinitely adjustable augment angle baseplate 102 connected to the implant 108 and the second bone 110.

The infinitely adjustable augment angle baseplate 102 can be configured to adjust an augment angle to alter a fit of the infinitely adjustable augment angle baseplate 102 on a resected surface of a bone (e.g., the first bone 104, the second bone 110, or the like). The infinitely adjustable augment angle baseplate 102 can be insertable into the implant 108 and can be configured to receive the engagement member 112. As such, the infinitely adjustable augment angle baseplate 102 can be configured to absorb energy from the engagement member 112 and distribute the absorbed energy throughout the implant 108. The infinitely adjustable augment angle baseplate 102 will be discussed in more detail with reference to FIG. 1-FIG. 7.

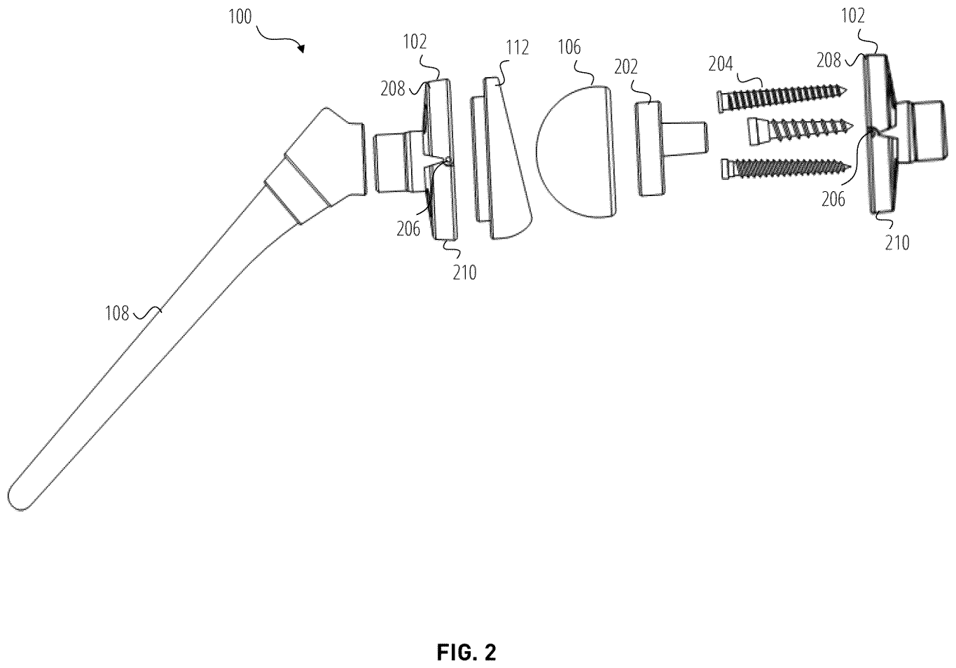

FIG. 2 illustrates an exploded view of an example of a prosthetic implant 100 including the infinitely adjustable augment angle baseplate 102. As shown in FIG. 2, the prosthetic implant 100 can include a taper adapter 202 and fasteners 204.

The taper adapter 202 can be configured to be inserted into the infinitely adjustable augment angle baseplate 102 and can couple to the artificial head component 106. As such, the taper adapter 202 can be configured to absorb energy from the artificial head component 106 and distribute that energy into the infinitely adjustable augment angle baseplate 102. In examples, the artificial head component 106 and the taper adapter 202 can be a single integral part. In other examples, such as the example shown in FIG. 2, the artificial head component 106 and the taper adapter 202 can be separate components that are configured to be coupled together.

The fasteners 204 can be configured to be inserted through the infinitely adjustable augment angle baseplate 102 to attach the infinitely adjustable augment angle baseplate 102 to the first bone 104 (FIG. 1). As shown in FIG. 2, the fasteners 204 can be screws of varying diameters and lengths to ensure solid attachment of the infinitely adjustable augment angle baseplate 102 and the bone (e.g., the first bone 104 (FIG. 1)). In examples, the fasteners 204 can be posts, pins, bolts, or any other fastener that can be used to attach the infinitely adjustable augment angle baseplate 102 to the bone of the patient.

The infinitely adjustable augment angle baseplate 102 can include a pivot connection 206. The pivot connection 206 can be configured to pivotably connect a first portion 208 and a second portion 210. The pivot connection 206 can include a hinged connection or a living spring connection, including bendable or flexible material to adjust the augment angle of the infinitely adjustable augment angle baseplate 102.

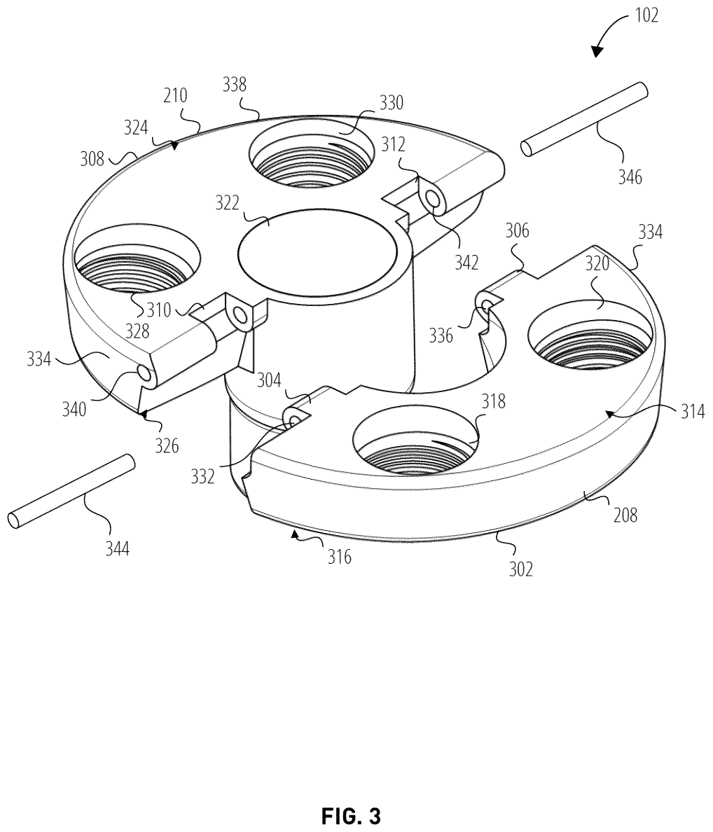

FIG. 3 illustrates an exploded view of an example of an infinitely adjustable augment angle baseplate 102. As discussed herein, the infinitely adjustable augment angle baseplate 102 can be configured to be a component of a prosthetic implant. As shown in FIG. 3, each of the first portion 208 and the second portion 210 can include a semi-circular profile 302 and a semi-circular profile 308, respectively.

The first portion 208 can include a first support surface 314, a first anchoring surface 316 opposite the first support surface 314, and each of a first fastener aperture 318 and a first fastener aperture 320 extending through the first portion 208 from the first support surface 314 to the first anchoring surface 316.

The first support surface 314 can be configured to provide support to the artificial head component 106 (FIG. 1), the taper adapter 202 (FIG. 2), the engagement member 112 (FIG. 1), or any other component of the prosthetic implant 100 attached to the infinitely adjustable augment angle baseplate 102. The first anchoring surface 316 can be configured to be adjacent the bone of the patient when the infinitely adjustable augment angle baseplate 102 is installed in the bone (e.g., either of the first bone 104 or the second bone 110).

Each of the first fastener aperture 318 and the first fastener aperture 320 can be configured to receive one or more fasteners to secure the infinitely adjustable augment angle baseplate 102 to the bone of the patient (e.g., the first bone 104, the second bone 110, or any other bone of a patient around a joint that can receive a prosthetic implant).

The second portion 210 can include a boss 322, a second support surface 324, a second anchoring surface 326 opposite the second support surface 324, and each of a second fastener aperture 328 and a second fastener aperture 330 extending through the second portion 210 from the second support surface 324 to the second anchoring surface 326.

As shown in FIG. 3, the boss 322 can include a female taper, which can be configured to receive one or more components of the prosthetic implant 100, such as the artificial head component 106 or the engagement member 112. Here, the boss 322 can extend from the second support surface 324 and past the second anchoring surface 326. In examples, the boss 322 can be configured to be inserted into the implant 108 to help secure the infinitely adjustable augment angle baseplate 102 to the implant 108 to provide support for the infinitely adjustable augment angle baseplate 102 and absorb energy from engagement between the artificial head component 106 and the engagement member 112.

In another example, the boss 322 can include a male tape such that the boss 322 extends from second support surface 324 away from the second anchoring surface 326. The boss 322 with the male taper can be insertable into one or more components of the prosthetic implant 100, such as the artificial head component 106 or the engagement member 112.

The second support surface 324 can be configured to provide support to the artificial head component 106 (FIG. 1), the taper adapter 202 (FIG. 2), the engagement member 112 (FIG. 1), or any other component of the prosthetic implant 100 attached to the infinitely adjustable augment angle baseplate 102. The second anchoring surface 326 can be configured to be adjacent the bone of the patient when the infinitely adjustable augment angle baseplate 102 is installed in the bone (e.g., either of the first bone 104 or the second bone 110).

Each of the second fastener apertures 328 and the second fastener aperture 330 can be configured to receive one or more fasteners to secure the infinitely adjustable augment angle baseplate 102 to the bone of the patient (e.g., the first bone 104, the second bone 110, or any other bone of a patient around a joint that can receive a prosthetic implant).

To support the pivot connection 206 each of the first portion 208 and the second portion 210 can include features. For example, the first portion 208 can include a first protrusion 304 and a second protrusion 306. The first protrusion 304 can include a first attachment aperture 332 and the second protrusion 306 can include a second attachment aperture 336. Similarly, to support the pivot connection 206, the second portion 210 can include a first cutout 310 and a second cutout 312. The second portion 210 can also include a third attachment aperture 340 extending from the perimeter 338 of the second portion 210 to the first cutout 310 and a fourth attachment aperture 342 extending from the perimeter 338 to the second cutout 312.

To facilitate the pivot connection 206, the first protrusion 304 and the second protrusion 306 can be complementary with the first cutout 310 and the second cutout 312, respectively. Also, to facilitate the pivot connection 206 the first attachment aperture 332, the second attachment aperture 336, the third attachment aperture 340, and the fourth attachment aperture 342 can be configured to be axially aligned as the first portion 208 is pivotably coupled to the second portion 210 via a first pin 344 being inserted within the third attachment aperture 340 and the first attachment aperture 332 and a second pin 346 being inserted into the fourth attachment aperture 342 and the second attachment aperture 336, respectively.

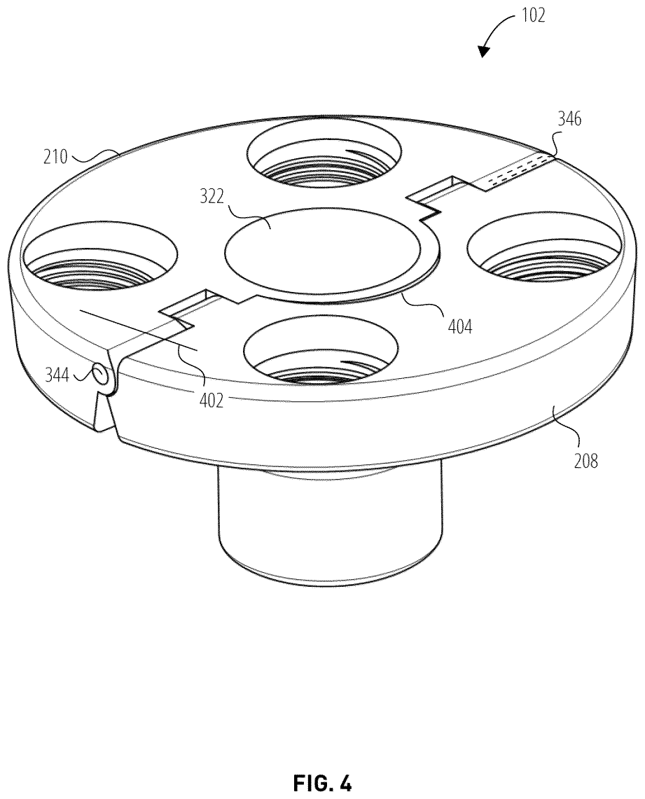

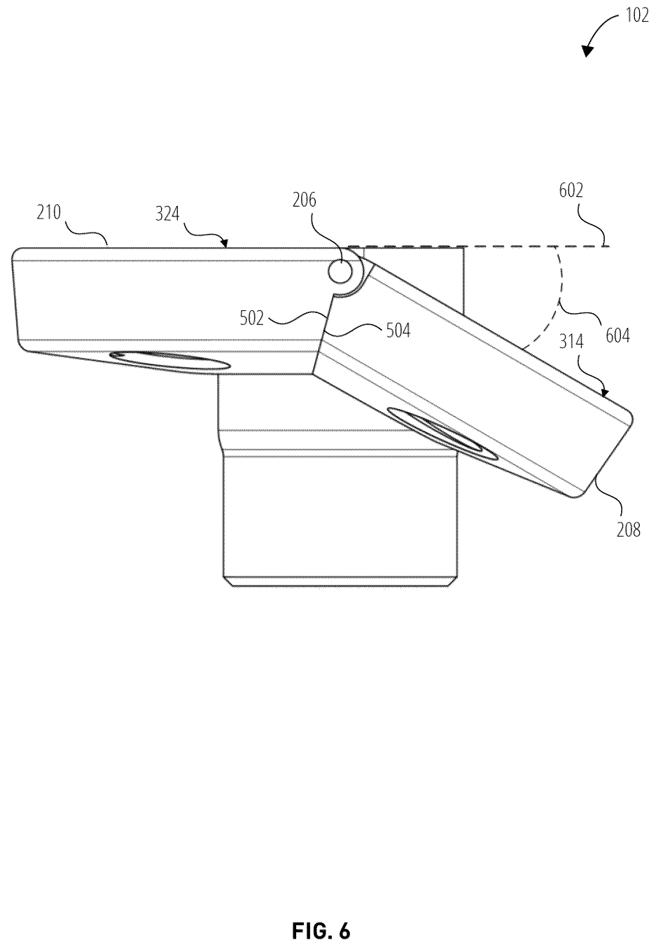

FIG. 4 illustrates a perspective view of an example of the infinitely adjustable augment angle baseplate 102. As discussed with reference to FIG. 2 and FIG. 3, the first portion 208 can be pivotably attached to the second portion 210 the pivot connection 206. Specifically, the first pin 344 and the second pin 346 can be used to pivotaly attach the first portion 208 and the second portion 210. Because of the pivot connection 206 the first portion 208 can pivot relative to the second portion 210 to change an augment angle 402 of the infinitely adjustable augment angle baseplate 102. The augment angle 402 can be between the first support surface 314 of the first portion 208 and a plane extending from the second portion 210 (the plane will be shown in FIG. 6).

As shown in FIG. 4, the first portion 208 can include an inner support surface 404, which can be configured to define a minimum of the augment angle 402 as the first portion 208 rotates relative to the second portion 210 about the pivot connection 206 (FIG. 2). For example, to limit the minimum of the augment angle 402, the inner support surface 404 can be configured to contact the boss 322 and stop further rotation of the first portion 208 relative to the second portion 210 about the pivot connection 206.

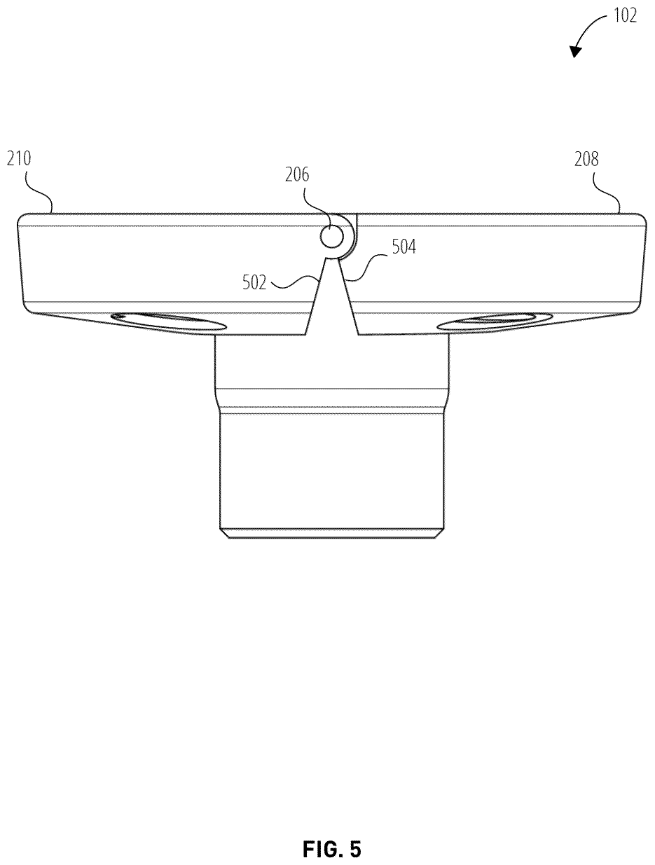

FIG. 5 illustrates a side view of an example of an infinitely adjustable augment angle baseplate 102. The first portion 208 can include a tapered portion 504 and the second portion 210 can include a tapered portion 502 to limit a maximum amount of rotation of the first portion 208 relative to the second portion 210 about the pivot connection 206. Either of the tapered portion 502 of the second portion 210 or the tapered portion 504 of the first portion 208 can be adjusted to change the maximum range of the augment angle 402 (first shown in FIG. 4).

For example, to increase the maximum range of the augment angle 402, either one of, or both of, the tapered portion 502 or the tapered portion 504 could be increased to allow more rotation of the first portion 208 about the pivot connection 206 before the tapered portion 502 and the tapered portion 504 engage one another to prevent further rotation about the pivot connection 206. To decrease the maximum range of the augment angle 402, either one of, or both of, the tapered portion 502 or the tapered portion 504 could be decreased to allow less rotation of the first portion 208 about the pivot connection 206 before the tapered portion 502 and the tapered portion 504 engage one another to prevent further rotation about the pivot connection 206.

FIG. 6 illustrates a side view of an example of the infinitely adjustable augment angle baseplate 102 showing an augment angle 604. The augment angle 604 can be an angle formed between a plane 602, which extends from the second support surface 324 of the second portion 210, and the first support surface 314 of the first portion 208. The augment angle 604 can be adjusted as the first portion 208 rotates relative to the second portion 210 about the pivot connection 206.

As shown in FIG. 6, a maximum value of the augment angle 604 can be defined by engagement between the tapered portion 502 and the tapered portion 504. Thus, the maximum value of the augment angle 604 can be adjusted by adjusting at least one of the tapered portion 502 or the tapered portion 504. In examples, the augment angle 604 can have a range from 0 degrees to 60 degrees. In examples, the augment angle 604 can have a range between 0 degrees and 45 degrees. In examples, the augment angle 604 can have a range between 0 degrees and 40 degrees.

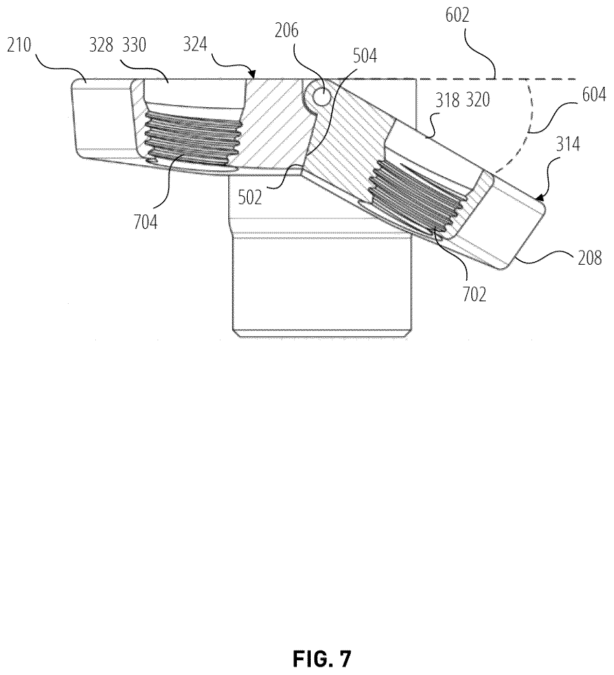

FIG. 7 illustrates a partial cross-sectional view of an example of infinitely adjustable augment angle baseplate 102 including the augment angle 604. FIG. 7 is an additional view that shows all the features discussed with reference to FIG. 6. Additionally, as shown in FIG. 7, the first fastener apertures (e.g., the first fastener aperture 318 or the first fastener aperture 320) and the second fastener apertures (e.g., the second fastener aperture 328 or the second fastener aperture 330) can include a first threaded surface 702 or a second threaded surface 704, respectively. The first threaded surface 702 and the second threaded surface 704 can help secure the infinitely adjustable augment angle baseplate 102 to the bone (e.g., the first bone 104 or the second bone 110) of the patient.

The following, non-limiting examples, detail certain aspects of the present subject matter to solve the challenges and provide the benefits discussed herein, among others.

Example 1 is a baseplate having an infinitely adjustable augment angle for a prosthetic implant, the baseplate comprising: a first portion including: a first support surface; a first anchoring surface opposite the first support surface; and a first fastener aperture extending through the first portion from the first support surface to the first anchoring surface; and a second portion pivotably attached to the first portion, the second portion including: a second support surface; a second anchoring surface opposite the second support surface; and a second fastener aperture extending through the second portion from the second support surface to the second anchoring surface; wherein pivoting the second portion relative to the first portion alters the infinitely adjustable augment angle.

In Example 2, the subject matter of Example 1 optionally includes wherein each of the first portion and the second portion include a semi-circular profile.

In Example 3, the subject matter of any one or more of Examples 1-2 optionally include wherein the infinitely adjustable augment angle comprises an angle formed between a plane extending from the first anchoring surface of the first portion and the second anchoring surface of the second portion.

In Example 4, the subject matter of Example 3 optionally includes wherein the second portion can be pivoted relative to the first portion such that the angle between the plane extending from the first anchoring surface of the first portion and the second anchoring surface of the second portion is adjustable between zero and forty degrees.

In Example 5, the subject matter of any one or more of Examples 1-4 optionally include wherein the first portion further includes a boss extending from the first anchoring surface.

In Example 6, the subject matter of Example 5 optionally includes wherein the first portion further includes a bore formed within the boss, the bore including a bore opening located on the first support surface that is configured to receive a mating element of the prosthetic implant.

In Example 7, the subject matter of any one or more of Examples 1-6 optionally include wherein the first portion further includes a third fastener aperture extending through the first portion from the first support surface to the first anchoring surface.

In Example 8, the subject matter of Example 7 optionally includes wherein the second portion further includes a fourth fastener aperture extending through the second portion from the second support surface to the second anchoring surface.

In Example 9, the subject matter of any one or more of Examples 1-8 optionally include wherein the first fastener aperture and the second fastener aperture comprise threaded apertures.

In Example 10, the subject matter of any one or more of Examples 1-9 optionally include wherein the baseplate comprises: a pivot connection to pivotably connect the first portion and the second portion.

In Example 11, the subject matter of Example 10 optionally includes wherein the pivot connection comprises: a first cutout and a second cutout, each of the first cutout and the second cutout formed in the first portion of the baseplate; and a first protrusion and a second protrusion, each of the first protrusion and the second protrusion formed in the second portion of the baseplate; and wherein the first protrusion and the second protrusion are complementary the first cutout and the second cutout, respectively.

In Example 12, the subject matter of Example 11 optionally includes wherein the first portion includes a first attachment aperture extending from a perimeter of the first portion to the first cutout and a second attachment aperture extending from the perimeter of the first portion to the second cutout, wherein the first protrusion includes a third attachment aperture and the second protrusion includes a fourth attachment aperture, and wherein the first attachment aperture, the second attachment aperture, the third attachment aperture, and the fourth attachment aperture are configured to be axially aligned as the first portion is coupled to the second portion.

In Example 13, the subject matter of Example 12 optionally includes a first pin insertable into the first attachment aperture and the third attachment aperture; and a second pin insertable into the second attachment aperture and the fourth attachment aperture, the first pin and the second pin pivotably couple the second portion and the first portion.

Example 14 is a system for an anatomical total shoulder arthroplasty or reverse total shoulder arthroplasty, the system comprising: an infinitely adjustable augment angle baseplate configured to be secured to a first bone of a patient; an implant insertable into a second bone of a patient; a baseplate insertable into the implant and configured to be attached to the second bone of the patient; an engagement member couplable to either of the infinitely adjustable augment angle baseplate or the baseplate; and a prosthetic head couplable to the other of the infinitely adjustable augment angle baseplate or the baseplate, the prosthetic head engageable with the engagement member to form a prosthetic joint between the first bone and the second bone.

In Example 15, the subject matter of Example 14 optionally includes wherein the infinitely adjustable augment angle baseplate comprises: a first portion including: a first support surface; a first anchoring surface opposite the first support surface; and a first fastener aperture extending through the first portion from the first support surface to the first anchoring surface; and a second portion pivotably attached to the first portion, the second portion including: a second support surface; a second anchoring surface opposite the second support surface; and a second fastener aperture extending through the second portion from the second support surface to the second anchoring surface.

In Example 16, the subject matter of Example 15 optionally includes wherein pivoting the second portion relative to the first portion alters the infinitely adjustable augment angle baseplate.

In Example 17, the subject matter of any one or more of Examples 15-16 optionally include wherein the infinitely adjustable augment angle baseplate comprises an angle formed between a plane extending from the first anchoring surface of the first portion and the second anchoring surface of the second portion.

In Example 18, the subject matter of any one or more of Examples 15-17 optionally include wherein the infinitely adjustable augment angle baseplate comprises: a pivot connection to pivotably connect the first portion and the second portion.

In Example 19, the subject matter of Example 18 optionally includes wherein the pivot connection comprises: a first cutout and a second cutout, each of the first cutout and the second cutout formed in the first portion of the infinitely adjustable augment angle baseplate; and a first protrusion and a second protrusion, each of the first protrusion and the second protrusion formed in the second portion of the infinitely adjustable augment angle baseplate; and wherein the first protrusion and the second protrusion are complementary the first cutout and the second cutout, respectively.

In Example 20, the subject matter of Example 19 optionally includes wherein the first portion includes a first attachment aperture extending from a perimeter of the first portion to the first cutout and a second attachment aperture extending from the perimeter of the first portion to the second cutout, wherein the first protrusion includes a third attachment aperture and the second protrusion includes a fourth attachment aperture, and wherein the first attachment aperture, the second attachment aperture, the third attachment aperture, and the fourth attachment aperture are configured to be axially aligned as the first portion is coupled to the second portion, and wherein the infinitely adjustable augment angle baseplate comprises: a first pin insertable into the first attachment aperture and the third attachment aperture; and a second pin insertable into the second attachment aperture and the fourth attachment aperture, the first pin and the second pin pivotably couple the second portion and the first portion.

Example 21 is a system, method, or apparatus combining any element of any of Examples 1-20.

The above-detailed description includes references to the accompanying drawings, which form a part of the detailed description. The drawings show, by way of illustration, specific examples that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other examples may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

What is claimed is:

1. A baseplate having an infinitely adjustable augment angle for a prosthetic implant, the baseplate comprising:
a first portion including:
a first support surface;
a first anchoring surface opposite the first support surface; and
a first fastener aperture extending through the first portion from the first support surface to the first anchoring surface; and
a second portion pivotably attached to the first portion, the second portion including:
a second support surface;
a second anchoring surface opposite the second support surface; and
a second fastener aperture extending through the second portion from the second support surface to the second anchoring surface;
wherein pivoting the second portion relative to the first portion alters the infinitely adjustable augment angle.

2. The baseplate of claim 1, wherein each of the first portion and the second portion include a semi-circular profile.

3. The baseplate of claim 1, wherein the infinitely adjustable augment angle comprises an angle formed between a plane extending from the first anchoring surface of the first portion and the second anchoring surface of the second portion.

4. The baseplate of claim 3, wherein the second portion can be pivoted relative to the first portion such that the angle between the plane extending from the first anchoring surface of the first portion and the second anchoring surface of the second portion is adjustable between zero and forty degrees.

5. The baseplate of claim 1, wherein the first portion further includes a boss extending from the first anchoring surface.

6. The baseplate of claim 5, wherein the first portion further includes a bore formed within the boss, the bore including a bore opening located on the first support surface that is configured to receive a mating element of the prosthetic implant.

7. The baseplate of claim 1, wherein the first portion further includes a third fastener aperture extending through the first portion from the first support surface to the first anchoring surface.

8. The baseplate of claim 7, wherein the second portion further includes a fourth fastener aperture extending through the second portion from the second support surface to the second anchoring surface.

9. The baseplate of claim 1, wherein the first fastener aperture and the second fastener aperture comprise threaded apertures.

10. The baseplate of claim 1, wherein the baseplate comprises:
a pivot connection to pivotably connect the first portion and the second portion.

11. The baseplate of claim 10, wherein the pivot connection comprises:
a first cutout and a second cutout, each of the first cutout and the second cutout formed in the first portion of the baseplate; and
a first protrusion and a second protrusion, each of the first protrusion and the second protrusion formed in the second portion of the baseplate, the first protrusion and the second protrusion complementary to the first cutout and the second cutout, respectively.

12. The baseplate of claim 11, wherein the first portion includes a first attachment aperture extending from a perimeter of the first portion to the first cutout and a second attachment aperture extending from the perimeter of the first portion to the second cutout, wherein the first protrusion includes a third attachment aperture and the second protrusion includes a fourth attachment aperture, and wherein the first attachment aperture, the second attachment aperture, the third attachment aperture, and the fourth attachment aperture are configured to be axially aligned as the first portion is coupled to the second portion.

13. The baseplate of claim 12, comprising:
a first pin insertable into the first attachment aperture and the third attachment aperture; and
a second pin insertable into the second attachment aperture and the fourth attachment aperture, the first pin and the second pin pivotably couple the second portion and the first portion.

14. A system for an anatomical total shoulder arthroplasty or reverse total shoulder arthroplasty, the system comprising:
an infinitely adjustable augment angle baseplate configured to be secured to a first bone of a patient;
an implant insertable into a second bone of a patient;
a baseplate insertable into the implant and configured to be attached to the second bone of the patient;
an engagement member couplable to either of the infinitely adjustable augment angle baseplate or the baseplate; and
a prosthetic head couplable to the other of the infinitely adjustable augment angle baseplate or the baseplate, the prosthetic head engageable with the engagement member to form a prosthetic joint between the first bone and the second bone.

15. The system of claim 14, wherein the infinitely adjustable augment angle baseplate comprises:
a first portion including:
a first support surface;
a first anchoring surface opposite the first support surface; and
a first fastener aperture extending through the first portion from the first support surface to the first anchoring surface; and
a second portion pivotably attached to the first portion, the second portion including:
a second support surface;
a second anchoring surface opposite the second support surface; and
a second fastener aperture extending through the second portion from the second support surface to the second anchoring surface.

16. The system of claim 15, wherein pivoting the second portion relative to the first portion alters the infinitely adjustable augment angle baseplate.

17. The system of claim 15, wherein the infinitely adjustable augment angle baseplate comprises an angle formed between a plane extending from the first anchoring surface of the first portion and the second anchoring surface of the second portion.

18. The system of claim 15, wherein the infinitely adjustable augment angle baseplate comprises:
a pivot connection to pivotably connect the first portion and the second portion.

19. The system of claim 18, wherein the pivot connection comprises:
a first cutout and a second cutout, each of the first cutout and the second cutout formed in the first portion of the infinitely adjustable augment angle baseplate; and
a first protrusion and a second protrusion, each of the first protrusion and the second protrusion formed in the second portion of the infinitely adjustable augment angle baseplate, the first protrusion and the second protrusion complementary to the first cutout and the second cutout, respectively.

20. The system of claim 19, wherein the first portion includes a first attachment aperture extending from a perimeter of the first portion to the first cutout and a second attachment aperture extending from the perimeter of the first portion to the second cutout, wherein the first protrusion includes a third attachment aperture and the second protrusion includes a fourth attachment aperture, and wherein the first attachment aperture, the second attachment aperture, the third attachment aperture, and the fourth attachment aperture are configured to be axially aligned as the first portion is coupled to the second portion, and wherein the infinitely adjustable augment angle baseplate comprises:
a first pin insertable into the first attachment aperture and the third attachment aperture; and
a second pin insertable into the second attachment aperture and the fourth attachment aperture, the first pin and the second pin pivotably couple the second portion and the first portion.
